(19) United States
(12) Patent Application Publication
(10) Pub. No.: US 2015/0307519 A1
(43) Pub. Date: Oct. 29, 2015

(54) SMALL MOLECULES FOR RESTORING FUNCTION TO P53 CANCER MUTANTS

(71) Applicant: THE REGENTS OF THE UNIVERSITY OF CALIFORNIA, Oakland, CA (US)

(72) Inventors: Hartmut Luecke, Irvine, CA (US); Brad Wallentine, Irvine, CA (US); Chiung-Kuang Chen, Irvine, CA (US); Roberta Baronio, Irvine, CA (US); Peter Kaiser, Irvine, CA (US)

(73) Assignee: THE REGENTS OF THE UNIVERSITY OF CALIFORNIA, Oakland, CA (US)

(21) Appl. No.: 14/698,583

(22) Filed: Apr. 28, 2015

Related U.S. Application Data
(60) Provisional application No. 61/985,687, filed on Apr. 29, 2014.

(51) Int. Cl. C07D 513/14; C07D 277/46; C07C 235/66; C07D 231/40; C07C 275/40; C07D 487/04; C07D 235/02

(52) U.S. Cl. CPC C07D 513/14; C07D 487/04; C07D 277/46; C07D 235/02; C07D 231/40; C07C 275/40; C07C 235/66

(57) ABSTRACT

Embodiments of the present provide methods of inducing p53 SRMMP-dependent cancer cell cytotoxicity. Such methods involve the steps of exposing a cancer cell that comprises a p53 SRMMP to an amount of a chemical compound sufficient to induce p53 SRMMP-dependent cancer cell cytotoxicity. In one embodiment, the present invention provides a method of treating cancer by determining the presence of a p53 mutation, and administering a therapeutically effective dosage of a composition comprising one or more promoting compounds.

### CROSS-REFERENCE TO RELATED APPLICATIONS

The present application claims the benefit of priority under 35 U.S.C. §119(e) of U.S. Provisional Application Ser. No. 61/985,687, filed Apr. 29, 2014, the contents of which are hereby incorporated by reference.

### GOVERNMENT RIGHTS

This invention was made in part with United States Government support under Grant No. CA-112560, awarded by the National Institutes of Health. The U.S. Government has certain rights in this invention.

### REFERENCE TO SEQUENCE LISTING

The instant application contains a Sequence Listing which has been submitted in ASCII format via EFS-Web and is hereby incorporated by reference.

### FIELD OF THE INVENTIONS

Embodiments of the present invention relate to methods of isolating active conformation structural residue p53 missense mutant proteins found in cancers, assays for identifying compounds that promote maintenance of the active conformation of such p53 mutant protein, and small molecule compounds that promote maintenance of the active conformation of such p53 mutant protein.

### BACKGROUND OF THE INVENTIONS

Tumor suppressor protein p53 is a transcription factor that, in response to signals such as DNA damage, oncogene activation and hypoxia, controls apoptosis and cell cycle arrest pathways, among others. Tumor initiation and maintenance depend upon inactivation of p53 and therefor the pathways under its control that would otherwise deter uncontrolled cancer cell growth. Tumor suppressor protein p53 controls apoptosis and cell cycle arrest pathways through direct binding to p53 response elements in the promoters of target genes in those pathways. Such p53 response elements are related in sequence, but not identical. And p53 generally binds to p53 response elements in promoters of cell cycle arrest genes with high affinity and to p53 response elements in promoters of apoptotic genes with lower affinity.

Tumor suppressor protein p53 is a homotetramer, each chain of which is composed of a transactivation domain located in the N-terminal region, a DNA-binding core domain located in the central region, and tetramerization and regulatory domains located in the C-terminal region. Approximately 50% of all human cancers have mutant p53. And approximately 75% of such cancers have a single amino acid residue missense mutation in the DNA-binding core domain. The six most frequently mutated amino acid residues in p53's DNA-binding core domain are Arg-248, Arg-273, Arg-175, Gly-245, Arg-249 and Arg-282. The Arg-248 and Arg-273 residues are classified as contact residues because, in the wild-type p53 protein, they directly contact DNA. The Arg-175, Gly-245, Arg-249 and Arg-282 residues are classified as structural residues because, in the wild-type p53 protein, they play a role in maintaining the structural integrity of p53's DNA-binding surface.

p53 structural residue missense mutant proteins (SRMMPs) are destabilized relative to wild-type p53 protein, yet functional at lower temperatures. The temperature sensitive (TS) nature of p53 SRMMPs has severe implications for their folding state and functionality under physiological conditions. In particular, the DNA-binding core domain of wild-type p53 protein is only marginally stable under physiological conditions, having a melting temperature near mammalian body temperature (i.e., 36° C. to 44° C. depending on experimental approach). Accordingly, even slightly destabilized, TS p53 SRMMPs are largely unfolded and nonfunctional at 37° C. For instance, the most frequent p53 mutation in human cancers is the p53 SRMMP, R175H, in which a histidine (H) replaces the normal arginine (R) at amino acid position 175. The melting temperature of p53 R175H is destabilized by 10° C. compared to wild-type p53 protein, which results in p53 R175H mutant protein being largely misfolded at 37° C. and therefor nonfunctional at deterring cancer cell proliferation under physiological conditions.

### SUMMARY OF THE INVENTIONS

The presence of p53 SRMMPs in approximately one-third of all human cancers offers a targeted therapy opportunity for potential anti-cancer drugs of broad application. Drugs that would restore, in cancer cells comprising such p53 SRMMP, wild-type p53 protein functions of activating cell cycle arrest and/or apoptotic pathways would deter cancer cell proliferation, shrink tumor volume, and the like. Accordingly, certain embodiments of the present provide methods to promote a p53^{R175H} protein function in a cancer cell. Such methods involve a step of exposing the cancer cell to an amount of one or more chemical compound(s) effective to promote the p53^{R175H} function in the cancer cell. Also in such embodiments, the cancer cell comprises a p53^{R175H} protein, the p53^{R175H} function is at least one of an inhibition of proliferation of the cancer cell and an induction of apoptosis in the cancer cell, and each of the one or more chemical compound(s) comprises a molecular structure selected from the group consisting of:

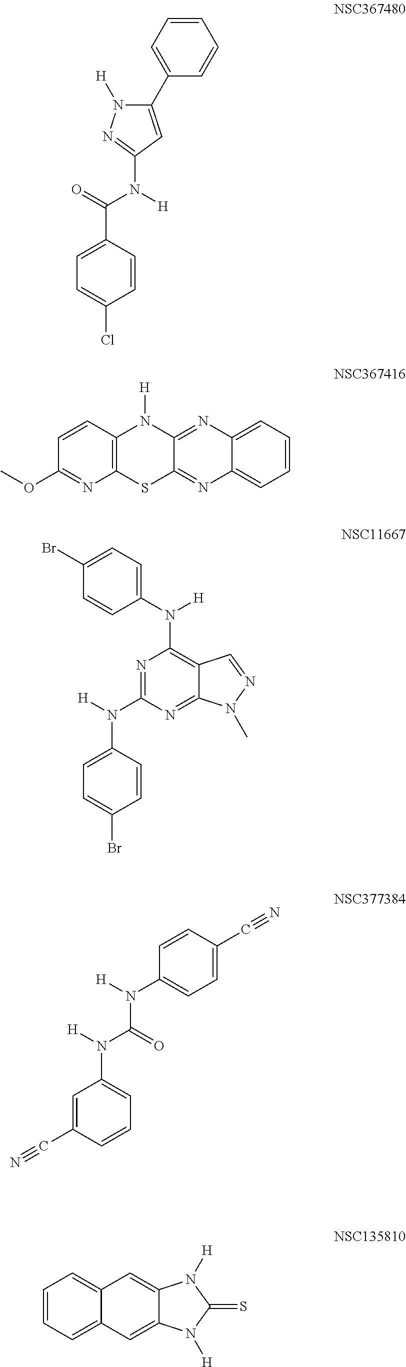 [SMILES: O=C(Nc1cc(-c2ccccc2)[nH]n1)c1ccc(Cl)cc1 ; COc1ccc2c(n1)Sc1nc3ccccc3nc1N2 ; Cn1ncc2c(Nc3ccc(Br)cc3)nc(Nc3ccc(Br)cc3)nc21 ; N#Cc1ccc(NC(=O)Nc2cccc(C#N)c2)cc1 ; S=c1[nH]c2cc3ccccc3cc2[nH]1]

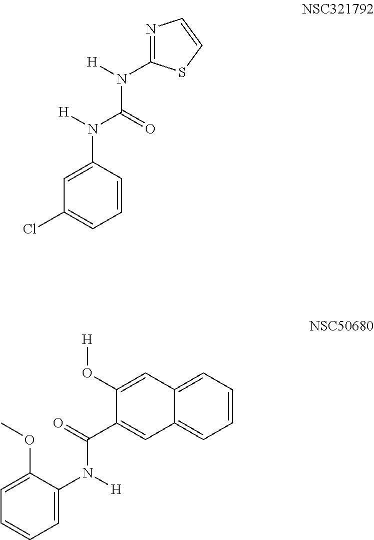 [SMILES: O=C(Nc1cccc(Cl)c1)Nc1nccs1 ; COc1ccccc1NC(=O)c1cc2ccccc2cc1O]

In some embodiments, the amount of each of the one or more chemical compound(s) effective to promote the p53^{R175H} function in the cancer cell is from about 10 μM to about 200 μM, preferably from about 20 μM to about 80 μM.

Certain embodiments of the invention provide methods to promote a p53^{G245S} protein function in a cancer cell. Such methods involve a step of exposing the cancer cell to an amount of one or more chemical compound(s) effective to promote the p53^{G245S} function in the cancer cell. Also in such embodiments, the cancer cell comprises a p53^{G245S} protein, the p53^{G245S} function is at least one of an inhibition of proliferation of the cancer cell and an induction of apoptosis in the cancer cell, and each of the one or more chemical compound(s) comprises a molecular structure selected from the group consisting of (wherein X is a halogen atom):

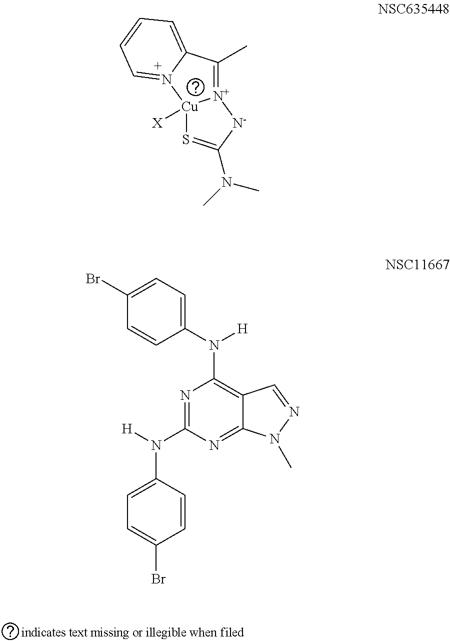

In some embodiments, the amount of the one or more chemical compound(s) effective to promote the p53^{G245S} function in the cancer cell is: i. from about 0.1 μM to about 100 μM for NSC635448, and/or ii. from about 20 μM to about 300 μM for NSC1167. In some embodiments, the amount of the one or more chemical compound(s) effective to promote the p53^{G245S} function in the cancer cell is: i. from about 0.1 μM to about 100 μM for NSC635448, and/or ii. from about 50 μM to about 100 μM for NSC1167.

In some embodiments, X is Br or Cl.

### BRIEF DESCRIPTION OF THE DRAWINGS

FIG. 1 shows, in accordance with various embodiments herein, the chemical structures of eight small chemical compounds that stabilized p53 R175H DBD protein by increasing its Tₘ in the DSF SLS assay described in Example 2 and promoted wild type p53 activity by p53 R175H in Saos-2 cell viability assays described in Examples 3 and 4.

FIG. 2, in accordance with various embodiments herein, is a plot of p53 R175H DBD protein stabilization as a function of exposure to 20 μM, 40 μM, 60 μM, or 80 μM of fourteen chemical compounds identified in the small chemical compound library screening described in Example 2.

FIG. 3, in accordance with various embodiments herein, is a plot. FIG. 3A is a plot of the survival of Saos-2 osteosarcoma cells devoid of p53 protein (light grey) and Saos-2 cells that express p53 R175H protein (dark grey) as a function of 72 hours of incubation with 0.1 μM, 0.5 μM, 1 μM, or 10 μM NSC635448, normalized to respective untreated cells. FIGS. 3B-3D are, respectively, plots of differential scanning fluorimetry determined Tₘs of p53 R175H DBD protein, wild-type p53 DBD protein, and chelated wild-type p53 DBD protein (4 μM) as a function of exposure to 20 μM, 40 μM, 60 μM, and 80 μM of either DMSO control (black), NSC635448 (red), NSC319725 (green), or NSC319725+CuCl₂ (orange).

FIG. 4, in accordance with various embodiments herein, is a plot of the relative number of Saos-2 p53 null cells (black) and Saos-2 cells that express p53 R175H protein (red) as a function of being cultured for three days in 50 μM PRIMA-1, 1 μM NSC635448, 50 μM NSC71948, 50 μM NSC367416, 100 μM NSC11667, 50 μM NSC152690, 50 μM NSC377384, 20 μM NSC13974, 100 μM NSC37433, 50 μM NSC135810, 50 μM NSC321792, 50 μM NSC50680, 40 μM NSC367480, 20 μM NSC67690, or 10 μM NSC71669.

FIG. 5, in accordance with various embodiments herein, shows pictures of micrographs. FIG. 5A shows pictures of Saos-2 cells devoid of p53 protein (p 53 null), Saos-2 cells generated as described in Example 3 that express p53 R175H protein, and Saos-2 cells as described in Example 3 that express p53 G245S protein cultured for 27 hours with vehicle (DMSO), 20 μM NCS319725, 1 μM NCS635448, or 100 μM NCS11667. FIG. 5B, in accordance with various embodiments herein, is a plot of cell counts, normalized to p53 null Saos-2 cells, of Saos-2 cells devoid of p53 protein (p53 null), Saos-2 cells generated as described in Example 3 that express p53 R175H protein, and Saos-2 cells generated as described in Example 3 that express p53 G245S protein as a function of being cultured for 27 hours with NCS635448. FIG. 5C, in accordance with various embodiments herein, is a plot of cell counts, normalized to p53 null Saos-2 cells, of Saos-2 cells devoid of p53 protein (p53 null), Saos-2 cells generated as described in Example 3 that express p53 R175H protein, and Saos-2 cells as described in Example 3 that express p53 G245S protein as a function of being cultured with cultured for 27 hours with NSC11667.

FIG. 6, in accordance with various embodiments herein, is a plot of p53 R175H protein-dependent activation. FIG. 6A depicts a plot of p53 R175H protein-dependent activation of a luciferase reporter under the control of a p53 response element from the p21 promoter in Saos-2 cells as a function of being cultured for three days with 20 μM NCS319725, 20 μM NSC63448, or 10 μM NSC63448. FIG. 6B is a plot of p53 R175H protein-dependent activation of a luciferase reporter under the control of a p53 response element from the PUMA promoter in Saos-2 cells as a function of being cultured for three days with 20 μM NCS319725, 20 μM NSC63448, or 10 μM NSC63448.

FIG. 7, in accordance with various embodiments herein, is a plot of the relative number of Saos-2 p53 null cells (black) and Saos-2 cells that express p53 R175H protein (grey) as a function of being cultured for three days in 1 μM CuCl₂, 0.1 μM CuCl₂, 1 μM ZnCl₂, 0.1 μM ZnCl₂, 1 μM FeCl₂, 0.1 μM FeCl₂, 1 μM NSC635448, 0.1 μM NSC635448, 1 μM NSC319725, 0.1 μM 319725, 1 μM NSC319725, 0.1 μM 319725, 1 μM NSC319725 plus CuCl₂, 0.1 μM 319725 plus CuCl₂, 1 μM NSC319725 plus ZnCl₂, 0.1 μM 319725 ZnCl₂, 1 μM NSC319725 plus FeCl₂, and 0.1 μM 319725 FeCl₂.

FIG. 8, in accordance with various embodiments herein, is thermal stabilization of R175H DBD by select thiosemicarbazones. R175H DBD protein, at 4 μM, was used with compound concentrations of 8 and 20 μM (2:1 and 5:1 compound:protein concentrations). Copper chloride was added at equimolar concentrations as each thiosemicarbazone.

FIG. 9, in accordance with various embodiments herein, depicts a chemical representation of select thiosemicarbazones that, with the addition of copper, stabilize p53.

FIG. 10, in accordance with various embodiments herein, scaffold of p53-stabilizing small molecule, whereby:

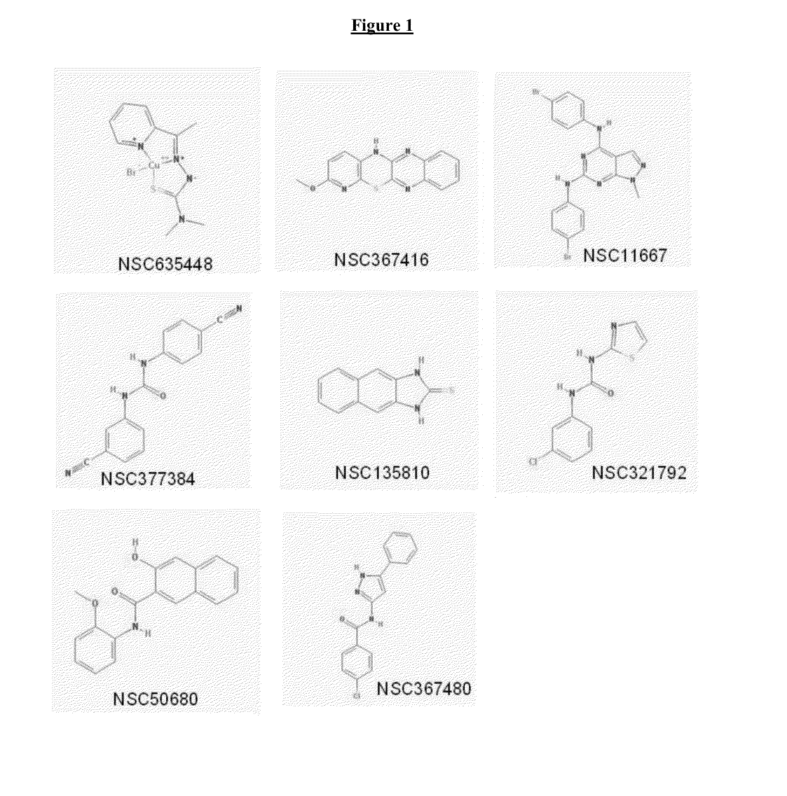

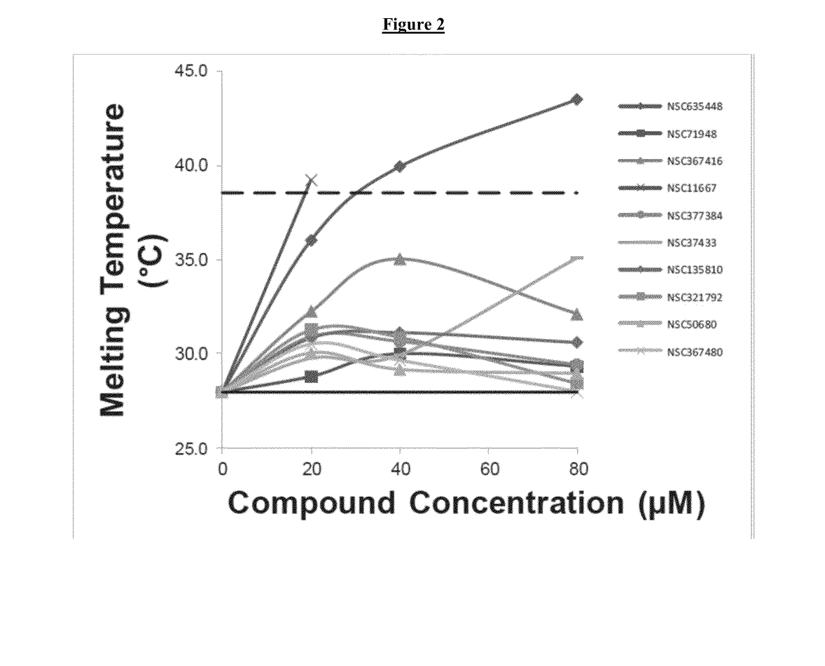

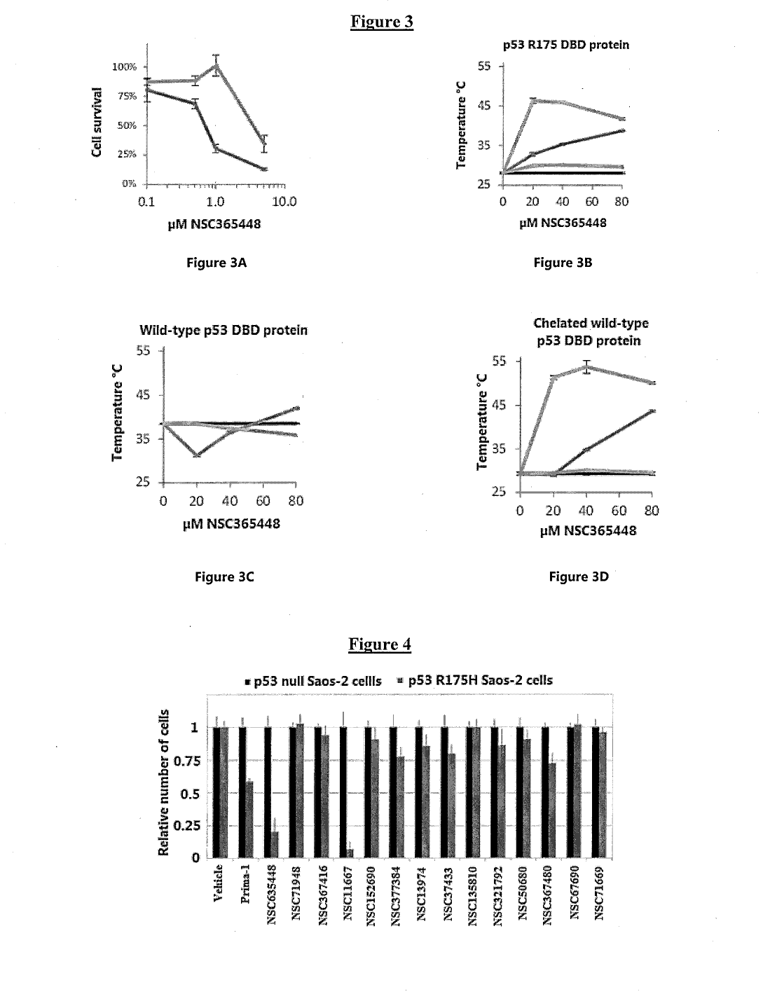

Q is either N or S; G11 is copper or another Group 11 element; R1-7 are independently H, halogen, alkyl, amide, hydroxyl, aryl, or hetercycl; Independent R1-7 substituents may together form an aryl or hetercycl.

### DETAILED DESCRIPTION OF THE INVENTIONS

Differential scanning fluorimetry (DSF) is a biophysical technique capable of identifying stabilizing or destabilizing physical interactions between protein and small chemical compounds by detecting changes in protein melting temperature (Tₘ) caused by such physical interactions. In DSF assays, small compounds that destabilize a protein decrease its Tₘ and small compounds that stabilize a protein increase its Tₘ. Small chemical compounds that stabilize p53 SRMMPs by physically interacting with them have broad potential as anti-cancer pharmaceuticals. This class of compounds could restore wild-type p53 protein functions of cell cycle arrest and apoptotic pathway activation in p53 SRMMP cancer cells because they have the potential to increase in such cells the population of active-configuration p53 SRMMP.

Four National Cancer Institute (NCI) small chemical compound libraries were screened in the DSF assay described in Example 2 for compounds that physically interact with a p53 SRMMP to the effect of stabilizing it. The NCI compound libraries were diversity set II (1579 compounds), approved oncology drugs set II (101 compounds), natural products set II (120 compounds), and mechanistic diversity set (879 compounds). The p53 SRMMP used in the DSF small chemical compound library screening (SLS) was the DNA binding core domain (DBD) of p53 R175H. The p53 R175 DBD was selected for several reasons. One reason is that p53 R175H is the most common p53 cancer mutation, comprising more than 5.0% of all missense mutations. Estimates state that 200,000 patients each year are diagnosed with cancers containing the p53 R175H mutation. Another reason is that p53 R175H creates a highly destabilized protein with a Tₘ of 28° C., about 10° C. below the Tₘ of wild-type p53. Such circumstances indicated that p53 R175H could provide a high-value translational research platform.

An aspect of the present invention is a method for expressing and isolating active-conformation p53 SRMMPs (e.g., p53 R175H DBD) in sufficient quantity and purity for use in SLS (see Example 1). The importance of this aspect of the invention derives from the thermodynamic instability of p53 SRMMPs (as reflected in their relatively lower Tₘ than wild-type p53 protein) and the requirement for properly folded p53 SMRRP for input into the DSF SLS assay described in Example 2. Since thermodynamic instability of the p53 R175H DBD leads to initial protein unfolding at about 14° C., the DSF SLS assay described in Example 2 was performed in a 4° C. cold room.

The DSF SLS assay described in Example 2 identified fifteen small chemical compounds in the aforementioned NCI libraries that stabilize p53 R175H DBD by at least 2° C. One such compound was not available from NCI in quantities sufficient for further characterization. The other fourteen compounds were obtained in milligram quantities and tested for their dose-dependent thermal stabilization of R175H DBD at 20 μM, 40 μM, and 80 μM. Six of the obtained compounds, NSC71948, NSC152690, NSC13974, NSC37433, NSC67690, and NSC71669, did not reproduce the stabilization of R175H DBD by more than 2° C. at 20 μM observed in the original screen. The balance of the obtained compounds stabilized R175H DBD between 2° C. and 4° C. at 20 μM. Two compounds stabilized R175H DBD over 10° C. within the 20 μM to 80 μM range. NSC635448 stabilized R175H DBD by more than 15° C., to 43.50±0.07° C. at 80 μM. At 20 μM, NSC11667 stabilized R175H DBD by about 11° C., to 39.22±0.13° C. At higher concentrations, NSC11667 alone produces temperature-dependent fluorescence and Tₘ measurements could not be confidently made for R175H DBD when NSC11667 was present at 40 μM or 80 μM. The addition of either NSC635448 (40 μM and 80 μM) or NSC11667 (20 μM) restores the thermodynamic stability of R175H DBD to the level of WT DBD.

The 14 candidate compounds identified as potentially possessing R175H DBD stabilizing properties were tested for their ability to reactivate p53^{R175H} in cells. These assays utilized human p53^{null} osteosarcoma Saos-2 cell lines engineered for doxycyclin-inducible expression of wild type (WT) and p53^{R175H}. Induced expression of p53^{WT} in these cells prevents proliferation and induces cell death, whereas induced expression of p53^{R175H} has no such effects. Compounds that restore activity to p53^{R175H} are expected to block cell proliferation and induce cell death in p53^{R175H} expressing cancer cells; but have no such effects on isogenic p53^{null} Saos-2 parent cells.

A dose range for each of PRIMA-1, a well characterized p53 reactivation compound, and the 14 candidate compounds identified in the R175H DBD thermal stabilization screen was first tested on the p53^{null} Soas-2 cells to determine the highest dose that does not affect Saos-2 cells proliferation (Cₘₐₓ). Saos-2 cells expressing p53^{R175H} and the p53^{null} parental cells were then cultured at the Cₘₐₓ doses for each compound, and cell counts were performed after 3 days. Reactivation of mutant p53 can be distinguished from general cytotoxic effects of compounds because only the latter block cell proliferation of both p53^{null} control cells and p53 mutant expressing cells. Control PRIMA-1 significantly reduced proliferation and survival to about 50% at its 50 μM Cₘₐₓ (FIG. 4). Most of the candidate compounds showed antiproliferative effects on p53^{R175H}-expressing Saos-2 cells (FIG. 4). Two of the candidate compounds had significant effects on p53^{R175H}-expressing Saos-2 cells without affecting p53^{null} cells (FIG. 4). NCS635448 reduced cell growth by 80% at a concentration of 1 μM and NSC11667 reduced cell proliferation by 90% at a concentration of 100 μM.

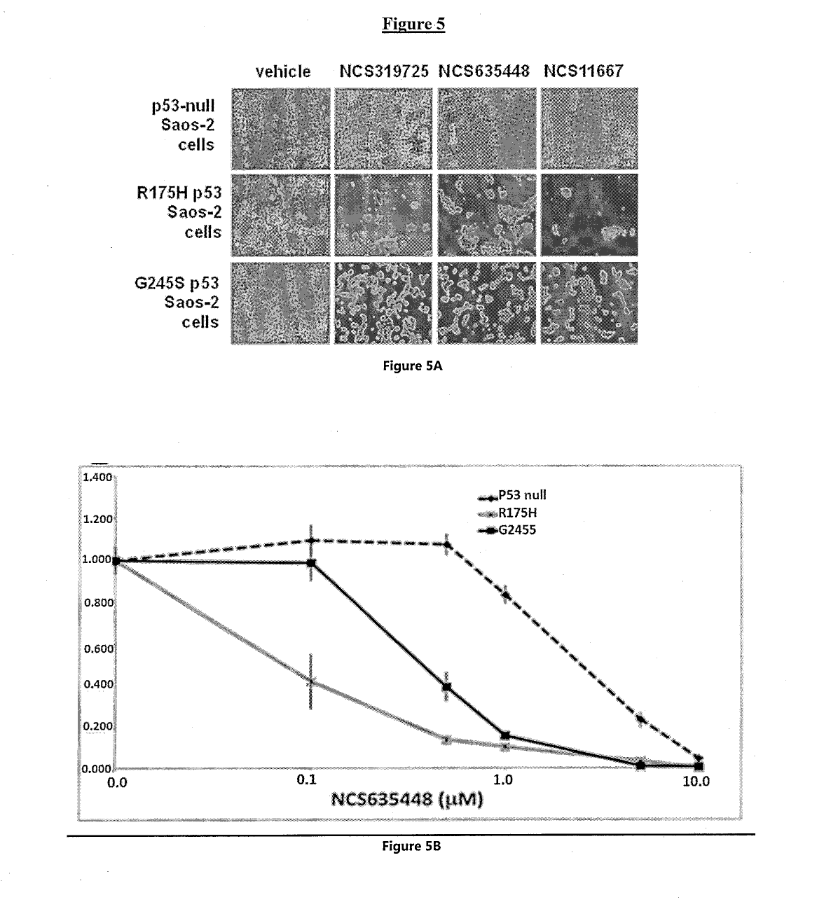

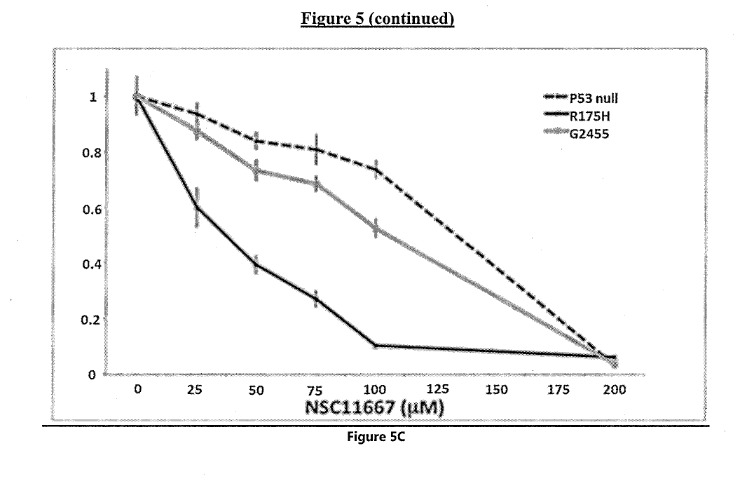

NCS635448 and NSC11667 were further tested for their ability to reactivate p53^{G245S}, the second most frequent p53 conformational missense mutation, in cancer cells. These assays utilized human p53^{null} Soas-2 cell lines engineered for doxycyclin-inducible expression of wild type (WT) and p53^{G245S} Induced expression of p53^{WT} in these cells prevents proliferation and induces cell death, whereas induced expression of p53^{G245S} has no such effects. Compounds that restore activity to p53^{G245S} are expected to block cell proliferation and induce cell death in p53^{G245S} expressing cells; but have no such effects on isogenic p53^{null} Soas-2 parent cells. In these experiments, NSC635448 and NSC11667 showed marked efficacy in reactivating p53^{G245S} (FIGS. 5A-5C).

NSC635448 belongs to a class of chemicals known as thiosemicarbazones. Thiosemicarbazones compound NSC319725 was recently reported as restoring p53 activity to cancer cells with p53^{R175H} mutations. (Yu et al. Allele-Specific p53 Mutant Reactivation. Cancer Cell 21(2012), pages 614-625.) NSC635448 differs from NSC319725 by the coordination of CuBr.

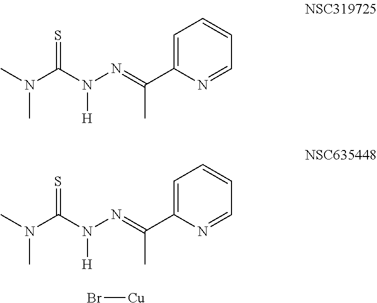

Thiosemicarbazones have a tridentate nitrogen-nitrogen-sulfur moiety known to allow the coordination of transition metals such as copper, iron, and zinc. Based on the structural similarities, the fact that NSC635448 is able to substantially stabilize R175H DBD, and recent results demonstrating antiproliferative effects of NSC319725 on p53^{R175H} cancer cell lines, it was suspected that NSC319725 could also increase the Tₘ of the p53 R175H DBD.

The thermodynamic stabilization of R175H DBD by NSC319725 was evaluated at 20 μM, 40 μM, and 80 μM concentrations in the above-described DSF assay (data not shown). Surprisingly, NSC319725 produced no stabilization of R175H. NSC319725 was then mixed with an equimolar concentration of CuCl₂ and again tested for R175H DBD stabilization using the DSF assay. NSC319725 plus CuCl₂ increased the Tₘ of R175H DBD by about 15° C. at 20 μM and 40 μM, surprisingly even more effectively than NSC635448. To determine whether this stabilization was specific for copper, NSC319725 was also tested after addition of Fe(II)Cl₂ and ZnCl₂. NSC319725 with Fe(II)Cl₂ did not stabilize R175H DBD; and addition of NSC319725 with ZnCl₂ to R175H DBD caused immediate precipitation. NSC635448, NSC319725, and NSC319725 were also tested for stabilization of G245S DBD and WT DBD in the DSF assay, yielding analogous results. NSC635448 increased the Tₘ of both proteins, and NSC319725 with CuCl₂ caused even greater stabilization. NSC319725 alone resulted in no effect or a slight destabilization.

In addition, the thermodynamic stability of R175H DBD was tested in the DSF assay with CuCl₂, Fe(II)Cl₂, and ZnCl₂ in the absence of a thiosemicarbazone. Surprisingly, both CuCl₂ and Fe(II)Cl₂ reduced the Tₘ of the protein, and addition of ZnCl₂ caused the R175H DBD to precipitate (data not shown).

NCS319725 and NCS319725 after the addition of equimolar concentrations of CuCl₂, FeCl₂, or ZnCl₂ were tested for their ability to reactivate p53^{R175H} in the above-described osteosarcoma cell-based assay (FIG. 7). Addition of copper did not enhance reactivation of p53^{R175H} in vivo over NCS319725. NSC319725 plus iron reduced the antiproliferative effects of NSC319725; and NSC319725 plus zinc increased general toxicity of NCS319725. The transition metals CuCl₂, ZnCl₂, or FeCl₂ alone showed substantially no effect on p53^{R175H} reactivation (FIG. 7).

FIG. 5A shows pictures of micrographs of Saos-2 cells devoid of p53 protein (vehicle), Saos-2 cells generated as described in Example 3 that express p53 R175H protein, and Saos-2 cells generated as described in Example 3 that express p53 G245S protein cultured for 27 hours with vehicle (DMSO), 20 μM NCS319725, 1 μM NCS635448, or 100 μM NCS11667. FIG. 5B is a plot of cell counts, normalized to p53 null Saos-2 cells, of Saos-2 cells devoid of p53 protein (p53 null), Saos-2 cells generated as described in Example 3 that express p53 R175H protein, and Saos-2 cells as described in Example 3 that express p53 G245S protein as a function of being cultured with cultured for 27 hours with 0 μM NCS635448, 0.1 μM NCS635448, 1 μM NCS635448, or 10 μM NCS635448. FIG. 5C is a plot of cell counts, normalized to p53 null Saos-2 cells, of Saos-2 cells devoid of p53 protein (p53 null), Saos-2 cells generated as described in Example 3 that express p53 R175H protein, and Saos-2 cells as described in Example 3 that express p53 G245S protein as a function of being cultured with cultured for 27 hours with 0 μM NSC11667, 20 μM NSC11667, 75 μM NSC11667, 100 μM NSC11667, or 200 μM NSC11667. The pictures and graphs in FIGS. 5A-5C indicate that NCS319725 and NCS635448 demonstrate marked p53 R175H- and p53 G245S-dependent cancer cell cytotoxicity.

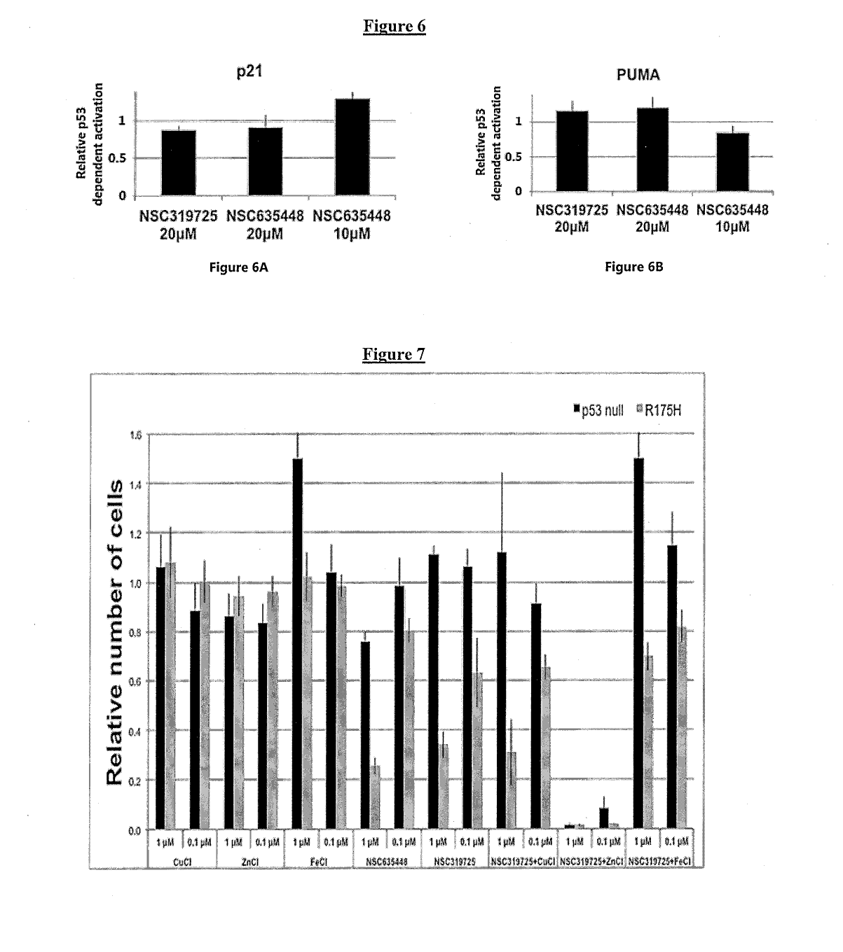

FIG. 6A is a plot of p53 R175H activation of a luciferase reporter under the control of a p53 response element from the p21 promoter in Saos-2 cells as a function of being cultured for three days with 20 μM NCS319725, 20 μM NSC63448, or 10 μM NSC63448. The Saos-2 cells were generated as described in Example 3; and not only express p53 R175H protein but also carry the p21 luciferase reporter construct described in Example 3. The luciferase reporter assays underlying the FIG. 4C plot were conducted as described in Example 5.

FIG. 6B is a plot of p53 R175H activation of a luciferase reporter under the control of a p53 response element from the PUMA promoter in Saos-2 cells as a function of being cultured for three days with 20 μM NCS319725, 20 μM NSC63448, or 10 μM NSC63448. The Saos-2 cells were generated as described in Example 3; and not only express full length p53 R175H protein but also carry the PUMA luciferase reporter construct described in Example 3. The luciferase reporter assays underlying the FIG. 4C plot were conducted as described in Example 5.

### Example 1

### Expression, Purification of Wild-Type and p53 R175H DBD Protein

Nucleic acid sequences coding for wild-type p53 protein DBD protein (residues 94-312 of the full length) had been previously introduced into the bacterial expression vector pSE420. From the wild-type p53 DBD pSE420 expression vector, a p53 R175 DBD expression vector was generated using a site-directed mutagenesis kit (Invitrogen, Grand Island, N.Y.). Exposure to IPTG induces cells carrying these vectors to express the wild-type and R175H p53 DBD, respectively. According, the wild type and R175H pSE420 expression vectors were transformed into Escherichia coli Rosetta 2 (DE3) cells. The transformed cells were grown in extra rich Terrific Broth media at 37° C. After these cell cultures reached an OD₆₀₀ of 1.0, the culture temperature was reduced to 15° C., and the expression of p53 R175H DNA binding domain was induced by adding IPTG to the Terrific Broth media to a concentration of 1 mM. The induced bacterial cells were grown for 40 hours at 15° C., harvested by centrifugation at 5,000 G, and frozen at −80° C. The frozen cells were resuspended and lysed by mild sonication. Active-conformation p53 R175H DBD protein and wild-type p53 DBD protein were separated from other bacterial cell components by ultra-centrifugation at 100,000 G and collecting the supernatant. From the collected supernatant. Active-conformation p53 R175H DBD protein and wild-type p53 DBD protein were each isolated to greater than 90% purity by conducting the following chromatographic steps: a SP-Sepharose cation exchange column, an affinity chromatography using a HiTrap heparin Sepharose column, and a gel filtration on a Superdex 200 column. This expression of purification scheme produces milligram amounts of p53 R175H DBD protein to execute SLS with the above-specified NCI structure diversity small chemical compound libraries.

### Example 2

### Differential Scanning Fluorimetry (DSF) and SLS

Purified p53 R175H DBD protein (0.2 mM in 20 mM HEPES at pH 7.4, 0.1 mM dithiothreitol (DTT), 150 mM NaCl) was diluted to 4 μM in 20 mM HEPES at pH 7.4. SLS compounds were dissolved in DMSO to specified concentrations and added to diluted p53 R175H DBD protein and then incubated for 30 minutes, all at 4° C. An equivalent volume of DMSO was used as a control. Lysozyme S100A4 and S100A10 were used as control proteins to rule out nonspecific interactions between SLS compounds and p53 R175H DBD protein. Sypro Orange stain (Bio-Rad, Hercules, Calif.) at 5,000× stock concentration was diluted to 5× concentration in 20 mM HEPES at pH 7.4 and added to each well of a 96-well plate. All SLS compounds were independently measured for fluorescence background to eliminate false positive results. Each protein (p53 R175H DBD, lysozyme S100A4 and S100A10) and SLS compound was DSF assayed in replicates of six in 50 μl per well in the 96-well plate, as well as with controls. DSF assays were also performed on wild-type p53 protein and p53 R175H DBD protein to establish the baseline Tₘ for those proteins. All DSF assays were performed with a Touch RT-PCR system CFX96 (Bio-Rad) with the starting temperature of 4° C. by increasing the temperature of the plate 1.0° C. per min to 94° C. Fluorescence measurements were taken once every minute during all experiments. Tₘ values for each well, mean Tₘ values, and 95% confidence intervals were calculated with the program Prism 5 (GraphPad Software, La Jolla, Calif.) by fitting the raw fluorescence data to a Boltzmann sigmoidal curve.

### Example 3

### Cell Lines

Soas-2 cells were obtained from the American Type Culture Collection (Manassas, Va., USA). They were cultured at 37° C. in DMEM high glucose supplemented with 10% FBS, penicillin G sodium (100 units ml⁻¹) and streptomycin (100 μg ml⁻¹). Stable Soas-2 cell lines harbouring doxycycline-inducible p53 wild type or mutants were established by a lentivirus-based strategy described in Metri, P. et al. MPK-09, a Small Molecule Inspired from Bioactive Styryllactone Restores the Wild-Type Function of Mutant p53. ACS chemical biology, doi:10.1021/cb3005929 (2013). Human p53 genes encoding specified p53 SRMMPs and wild-type p53 were cloned as SacI/PstI fragments into pEN_TmiRc3, replacing GFP and ccdB genes in the plasmid. Recombination of such p53-pEN_TmiRc3 vectors and the lentivirus vector pSlik was performed using the LR Clonase Enzyme Mix (Invitrogen, Carlsbad, Calif.) according to manufacturer's instructions. Lentiviruses were generated by co-transfecting 5 μg of lentiviral vector pSlik and 5 μg of each packaging vector (coding for Gag, Pol, Tat, Rev and VSVG) in 90% confluent 293T cells in 10-cm plates using Lipofectamine 2000 (Invitrogen, Carlsbad, Calif.). Supernatants were collected 48 h after transfection and used directly to infect Soas-2 cells. Forty-eight hours after this infection, the Soas-2 cultures were switched to hygromycin B-containing media. To isolate single clones expressing p53 SRMMP (p53^{R175H} or p53^{G245SS}) or wild-type p53, cells were induced with doxycycline and tested by immunoblotting. Doxycycline was used at a concentration of 1 μg ml⁻¹, hygromycin B at 50 μg ml⁻¹.

For generation of stable cell lines to measure p53 SRMMP-dependent activation of p21 or PUMA reporters, p21 (5′-GAAGAAGACTGGGCATGTCT-3′ SEQ. ID. NO.: 1) or PUMA (5′-CTGCAAGTCCTGACTTGTCC-3′ SEQ. ID. NO.: 2) response elements followed by a minimal promoter (5′-TAGAGGGTATATAATGGAAGCTCGACTTCCAG-3′ SEQ. ID. NO.: 3) were inserted into KpnI/XhoI cut plasmid pGL4.10 (Promega). These elements control the expression of a synthetic firefly luc2 (Photinus pyralis) gene. An SV40-driven puromycin resistance cassette was inserted into the Pst1 site of pGI4.10 to enable generation of stable cell lines. The PUMA and p21 reporter vectors were transfected into Soas-2 cells and single colonies resistant to 50 μg ml⁻¹ puromycin were selected.

### Example 4

### Cell Viability Assay

Cells generated as described in Example 3 were placed in 96-well plates at a density of 10,000 cells per well and incubated overnight. Doxycycline (final concentration 1 μg ml⁻¹) was then added to the cells to induce the expression of p53 SRMMP for 8 hours before solvent or p53 PRIMA-1 (final concentration 50 μM) or the specified NSC compounds were added to the cultures. PRIMA-1 was preheated to 100° C. for 10 min to promote formation of active decomposition products and then cooled before it was used in these experiments. Cell viability assays were performed using CellTiter-Glo Luminescent Cell Viability Assay (Promega, Wisconsin, Md.) according to the manufacturer's instructions. Data represent the average of four independent samples.

### Example 5

### PUMA and p21 Reporter Assay

Soas-2-PUMA-luc2 or Soas-2-p21-luc2 cell lines generated as described in Example 3 were transfected with 500 ng of cytomegalovirus-renilla plasmid and 125 ng of plasmid carrying either empty vector, p53 R175H, p53 G245S, or wild-type p53 expression cassettes using Lipofectamine 2000. The specified amounts of NSC319725 or NSC635488 were added to the cells 21 h after transfection. Activation of PUMA and p21 reporters in the cell lines was determined with a luciferase assay (Dual luciferase assay system, Promega) 8 hours after the addition of NSC319725 or NSC635488.

Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A method to promote a p53^{R175H} protein function in a cancer cell, the method comprising exposing the cancer cell to an amount of one or more chemical compound(s) effective to promote the p53^{R175H} function in the cancer cell, wherein:
the cancer cell comprises a p53^{R175H} protein;
the p53^{R175H} function is at least one member selected from the group consisting of an inhibition of proliferation of the cancer cell and an induction of apoptosis in the cancer cell; and
each of the one or more chemical compound(s) comprises a molecular structure selected from the group consisting of:

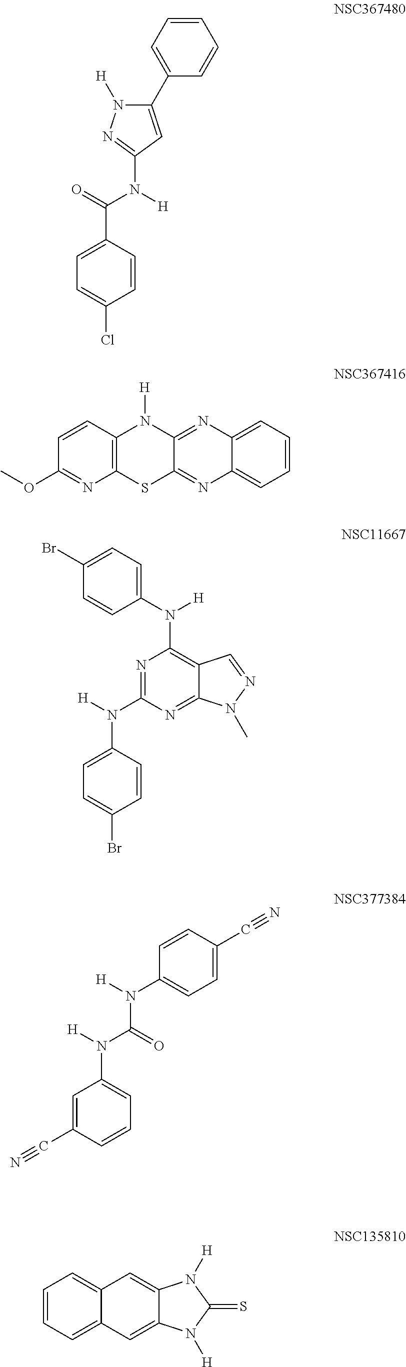 [SMILES: O=C(Nc1cc(-c2ccccc2)[nH]n1)c1ccc(Cl)cc1 ; COc1ccc2c(n1)Sc1nc3ccccc3nc1N2 ; Cn1ncc2c(Nc3ccc(Br)cc3)nc(Nc3ccc(Br)cc3)nc21 ; N#Cc1ccc(NC(=O)Nc2cccc(C#N)c2)cc1 ; S=c1[nH]c2cc3ccccc3cc2[nH]1]

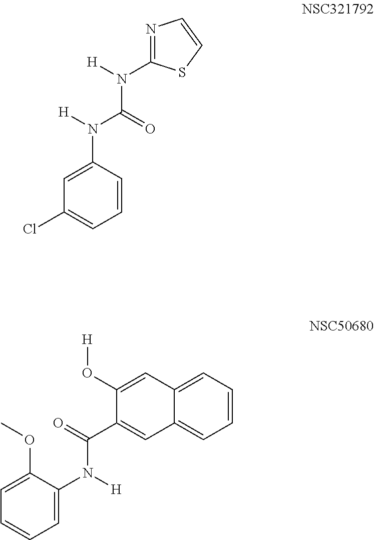 [SMILES: O=C(Nc1cccc(Cl)c1)Nc1nccs1 ; COc1ccccc1NC(=O)c1cc2ccccc2cc1O]

2. The method of claim 1, wherein the amount of each of the one or more chemical compound(s) effective to promote the p53^{R175H} function in the cancer cell is from about 10 μM to about 200 μM.

3. The method of claim 1, wherein the amount of each of the one or more chemical compound(s) effective to promote the p53^{R175H} function in the cancer cell is from about 20 μM to about 80 μM.

4. The method of claim 1, wherein the compound is NSC367416.

5. The method of claim 1, wherein the compound is NSC11667.

6. The method of claim 1, wherein the compound is NSC377384.

7. The method of claim 1, wherein the compound is NSC321792.

8. The method of claim 1, wherein the compound is NSC50680.

9. The method of claim 1, wherein the compound is NSC367480.

10. A method to promote a p53^{G245S} protein function in a cancer cell, the method comprising exposing the cancer cell to an amount of one or more chemical compound(s) effective to promote the p53^{G245S} function in the cancer cell, wherein:
the cancer cell comprises a p53^{G245S} protein;
the p53^{G245S} function is at least one member selected from the group consisting of an inhibition of proliferation of the cancer cell and an induction of apoptosis in the cancer cell; and
each of the one or more chemical compound(s) comprises a molecular structure selected from the group consisting of: 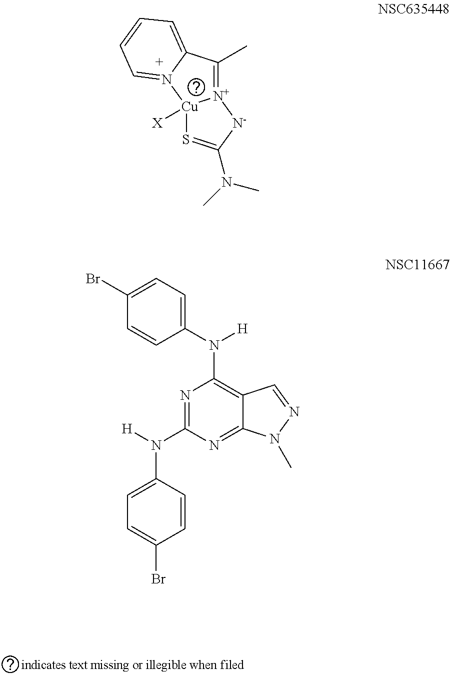
wherein X is a halogen atom.

11. The method of claim 10, wherein the amount of one or more chemical compound(s) effective to promote the p53^{G245S} function in the cancer cell is: i. from about 0.1 μM to about 100 μM for NSC635448, and/or ii. from about 20 μM to about 300 μM for NSC1167.

12. The method of claim 10, wherein the amount of the one or more chemical compound(s) effective to promote the p53^{G245S} function in the cancer cell is: i. from about 0.1 μM to about 100 μM for NSC635448, and/or ii. from about 50 μM to about 100 μM for NSC1167.

13. The method of claim 10, wherein X is Br or Cl.

14. A method of treatment of cancer in a subject, comprising:
obtaining a sample from the subject;
assaying the sample to determine the presence of a p53 mutation;
treating the subject with a therapeutically effective dosage of a composition comprising one or more promoting compounds.

15. The method of claim 14, wherein the p53 mutation is R174H.

16. The method of claim 14, wherein the p53 mutation is G245S.

17. The method of claim 14, wherein the one or more promoting compounds is one or more of the following:

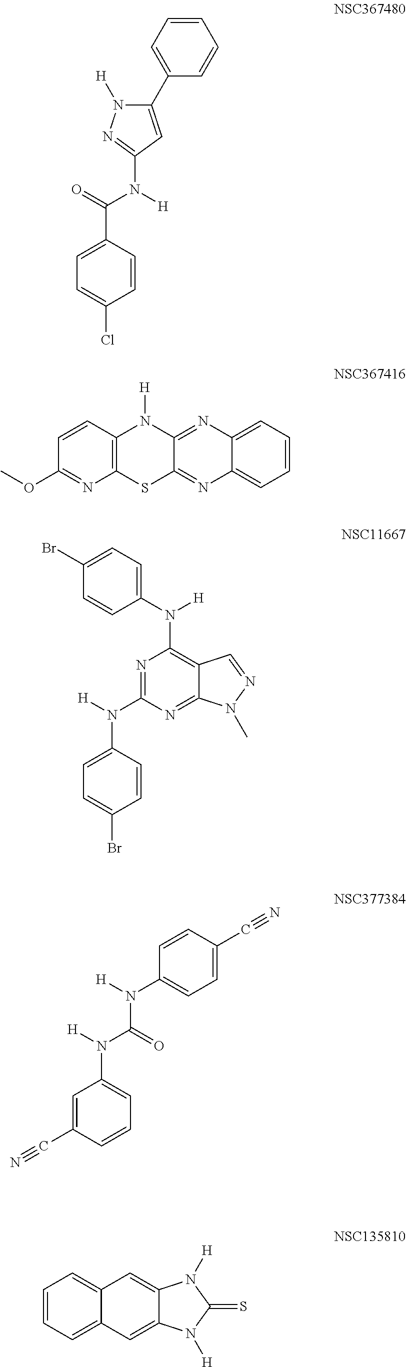 [SMILES: O=C(Nc1cc(-c2ccccc2)[nH]n1)c1ccc(Cl)cc1 ; COc1ccc2c(n1)Sc1nc3ccccc3nc1N2 ; Cn1ncc2c(Nc3ccc(Br)cc3)nc(Nc3ccc(Br)cc3)nc21 ; N#Cc1ccc(NC(=O)Nc2cccc(C#N)c2)cc1 ; S=c1[nH]c2cc3ccccc3cc2[nH]1]

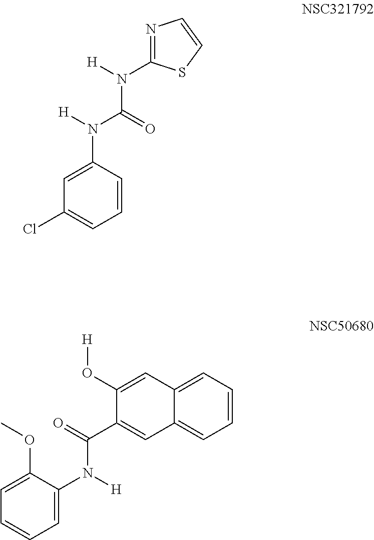 [SMILES: O=C(Nc1cccc(Cl)c1)Nc1nccs1 ; COc1ccccc1NC(=O)c1cc2ccccc2cc1O]

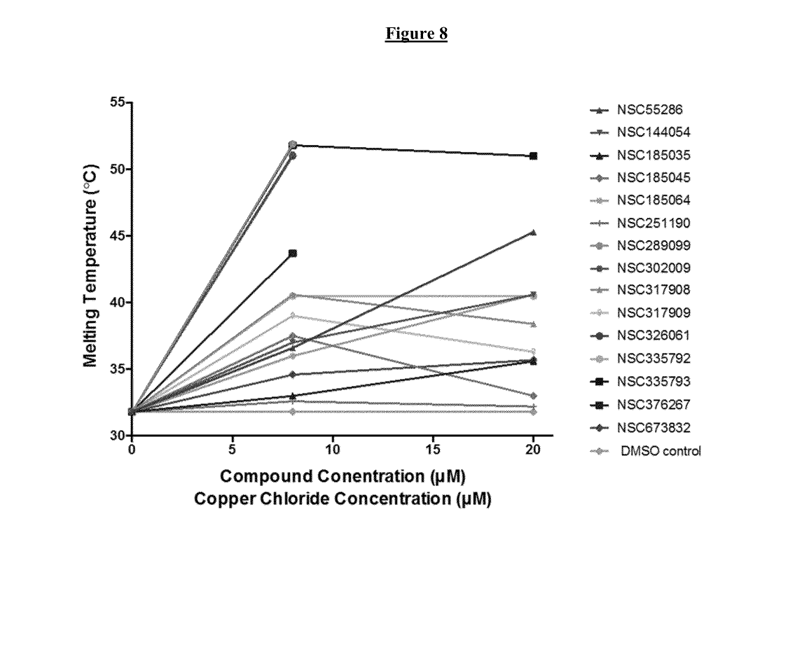

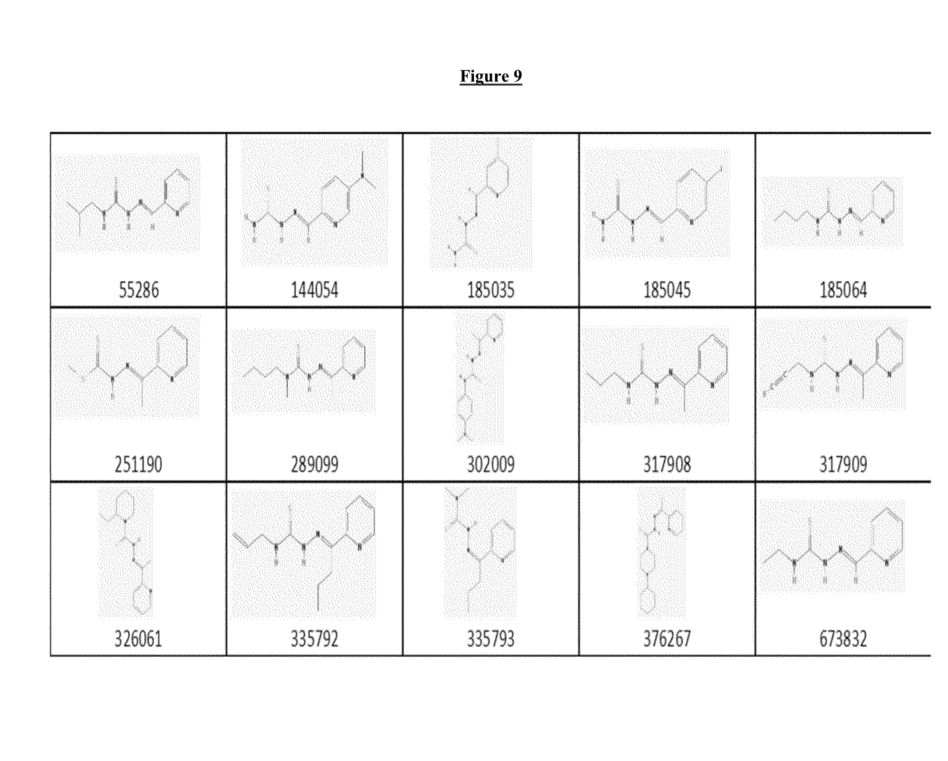

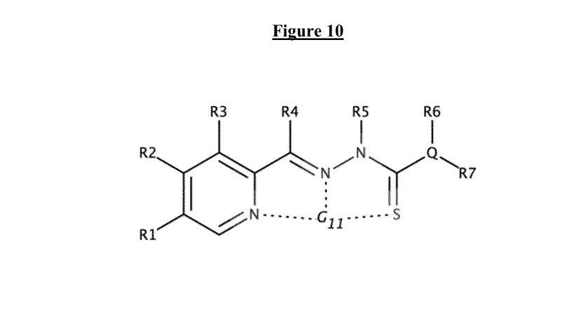

18. The method of claim 14, wherein the promoting compound is described in FIGS. 8, 9 and/or 10 herein.

19. The method of claim 14, wherein the promoting compound comprises a thiosemicarbazone.

20. The method of claim 14, wherein the promoting compound comprises a thioscemicarbazone and one or more transition metals.

21. A composition comprising a scaffold of one or more p53-stabilizing molecules.

22. The composition of claim 21, wherein the one or more p53-stabilizing molecules is described in FIG. 9.

23. The composition of claim 21, wherein the one or more p53-stabilizing molecules is described in FIG. 10, wherein Q is either N or S, G11 is copper or another Group 11 element, R1-7 are independently H, halogen, alkyl, amide, hydroxyl, aryl, or hetercycl, and Independent R1-7 substituents may together form an aryl or hetercycl.

24. The composition of claim 21, wherein the one or more p53-stabilizing molecules is selected from the group consisting of:

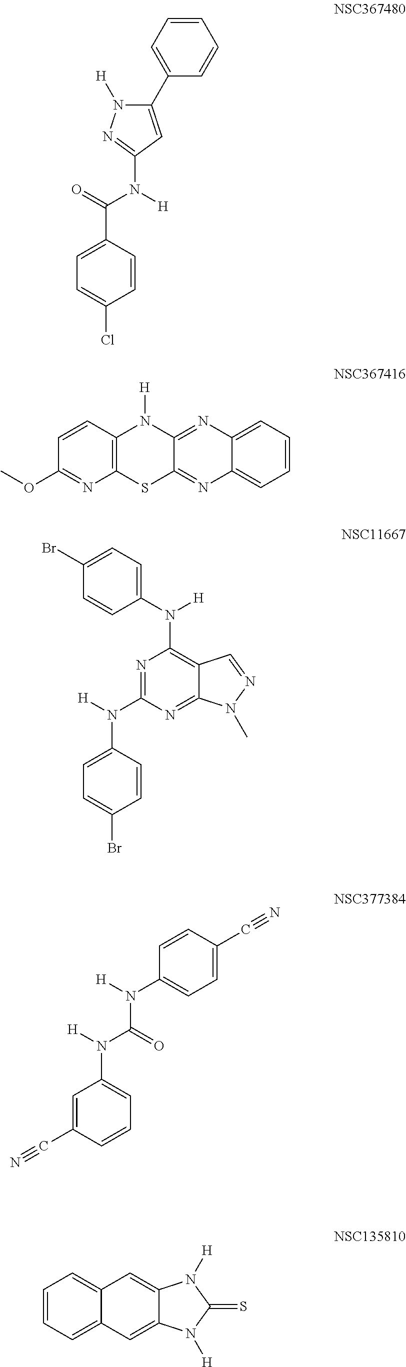 [SMILES: O=C(Nc1cc(-c2ccccc2)[nH]n1)c1ccc(Cl)cc1 ; COc1ccc2c(n1)Sc1nc3ccccc3nc1N2 ; Cn1ncc2c(Nc3ccc(Br)cc3)nc(Nc3ccc(Br)cc3)nc21 ; N#Cc1ccc(NC(=O)Nc2cccc(C#N)c2)cc1 ; S=c1[nH]c2cc3ccccc3cc2[nH]1]

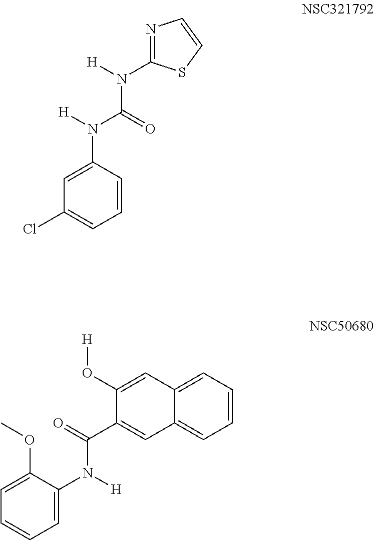 [SMILES: O=C(Nc1cccc(Cl)c1)Nc1nccs1 ; COc1ccccc1NC(=O)c1cc2ccccc2cc1O]

25. The composition of claim 21, wherein the one or more p53-stabilizing molecules is selected from the group consisting of: 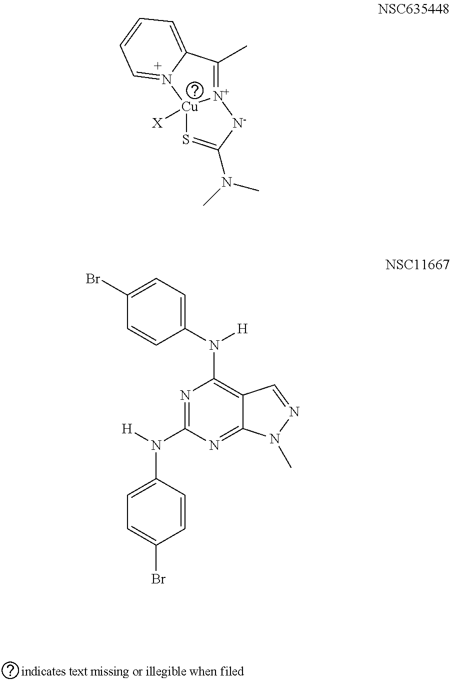
wherein X is a halogen atom.


SEQUENCE LISTING (3 sequences)

SEQ ID NO: 1 - DNA, 20 bp, Homo sapiens
gaagaagactgggcatgtct

SEQ ID NO: 2 - DNA, 20 bp, Homo sapiens
ctgcaagtcctgacttgtcc

SEQ ID NO: 3 - DNA, 32 bp, Homo sapiens
tagagggtatataatggaagctcgacttccag